(19) United States
(12) Patent Application Publication
(10) Pub. No.: US 2005/0101918 A1
(43) Pub. Date: May 12, 2005

(54) Protective injection needle

(76) Inventors: Chang-Tzu Chen, Taipei (TW); Chin-Ping Ting, Taoyuan Hsien (TW)

Correspondence Address: John G. Chupa;Law Offices of John Chupa & Associates, P.C., Suite 50, 28535 Orchard Lake Road, Farmington Hills, MI, 48334, US

(21) Appl. No.: 10/751,868

(22) Filed: Jan. 6, 2004

(30) Foreign Application Priority Data
Nov. 12, 2003 (TW) 092221768

(51) Int. Cl. A61M 5/32

(52) U.S. Cl. 604/192

(57) ABSTRACT

A protective injection needle comprising a sleeve with a sleeve front, a chamber is formed inside, a pair of blocking slots with a corresponding blocking piece face to face are on the inner center wall, a pair of none-penetrated fastening slots face to face are on the back of the inner wall, a pair of sleeve rings are on the front and back inner wall, a pair of none-penetrated stopping slots face to face are on the back inner wall, a sleeve path spans the two sleeve rings; a needle base fastens a needle with a needle stand and connects to the barrel, a none-penetrated base slot corresponding to the blocking slot is on the needle base, a fastening tenon corresponding to the fastening slot is on a flat fastening plane, while assembly, the blocking piece is inserted into the blocking slot, the fastening tenon also falls into the fastening slot, the blocking piece is in the base slot. Users connect the barrel and the needle stand for medicine, turn certain angle to have the fastening tenon fall into the sleeve path, push needle out from the sleeve front, then pull the plunger outward to suck medicine; after injection, the barrel and the plunger are pulled backward, the fastening also goes backward along the sleeve path, falls into the stopping slot and stops.

### BACKGROUND OF THE INVENTION

1. Field of the Invention

This invention relates generally to an injection needle and, more specifically, to a protective injection needle that prevents medical personnel from been damaged, infected disease and the reuse of the needle and needle base.

2. Description of the Prior Art

Heretofore, it is known that an injection needle is seen very often in medical treatment, doctors apply needles to draw medicines and inject these medicines into patients' body. Injection needles are essential for medical treatment.

The risk of medical treatments get higher and with the consideration of safety, the disposable needles become mainstream, especially after many medical personnel, such as nurses and doctors, damaged and infected disease by needles. Therefore they are very careful for needle injection, the choice of needles and to prevent reuse of needles become very important.

The known injection needle includes a barrel and a needle base connected to the barrel, and a plunger movable inside the barrel; in order not to expose the needle externally, a needle cap is applied; to prevent the needle from been reused, many different protective mechanisms are been developed.

### SUMMARY OF THE INVENTION

It is therefore a primary object of the invention to provide a protective injection needle for low cost, easy to operate and assemble, safe to apply.

In order to achieve the objective set forth, a protective injection needle in accordance with the present invention comprises a sleeve with a sleeve front, a chamber is formed inside, a pair of blocking slots with a corresponding blocking piece face to face are on the inner center wall, a pair of none-penetrated fastening slots face to face are on the back of the inner wall, a pair of sleeve rings are on the front and back inner wall, a pair of none-penetrated stopping slots face to face are on the back inner wall, a sleeve path spans the two sleeve rings, a needle base fastens a needle with a needle stand and connects to the barrel, a none-penetrated base slot corresponding to the blocking slot is on the needle base, a fastening tenon corresponding to the fastening slot is on a flat fastening plane, while assembly, the blocking piece is inserted into the blocking slot, the fastening tenon also falls into the fastening slot, the blocking piece is in the base slot. Users connect the barrel and the needle stand for medicine, turn certain angle to have the fastening tenon fall into the sleeve path, push needle out from the sleeve front, then pull the plunger outward to suck medicine; after injection, the barrel and the plunger are pulled backward, the fastening also goes backward along the sleeve path, falls into the stopping slot and stops.

### BRIEF DESCRIPTION OF THE DRAWINGS

The accomplishment of the above-mentioned object of the present invention will become apparent from the following description and its accompanying drawings which disclose illustrative an embodiment of the present invention, and are as follows:

FIG. 1 is an assembly view of the present invention;

FIG. 2 is a perspective view of a further embodiment of the present invention;

FIG. 3 is a cross-sectional view of the present invention;

FIG. 4 is another cross-sectional view of the present invention;

FIG. 5 is a cross-sectional view of a further embodiment in accordance with the present invention;

FIG. 6 is an application view the present invention;

FIG. 7 is another application view of the present invention; and

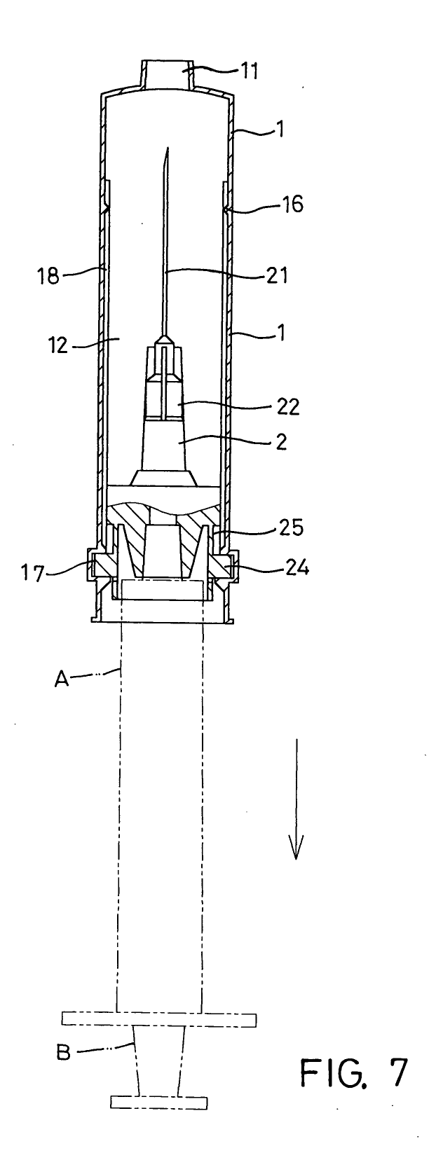

FIG. 8 is a cross-sectional view of a further embodiment in accordance with the present invention.

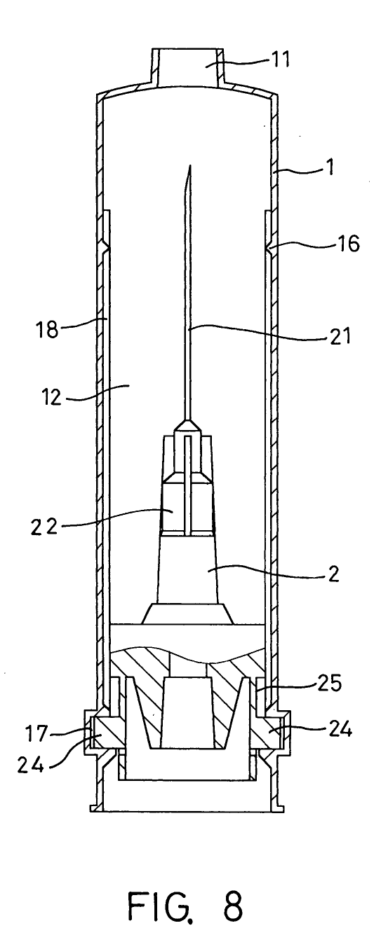

### DESCRIPTION OF THE PREFERRED EMBODIMENT

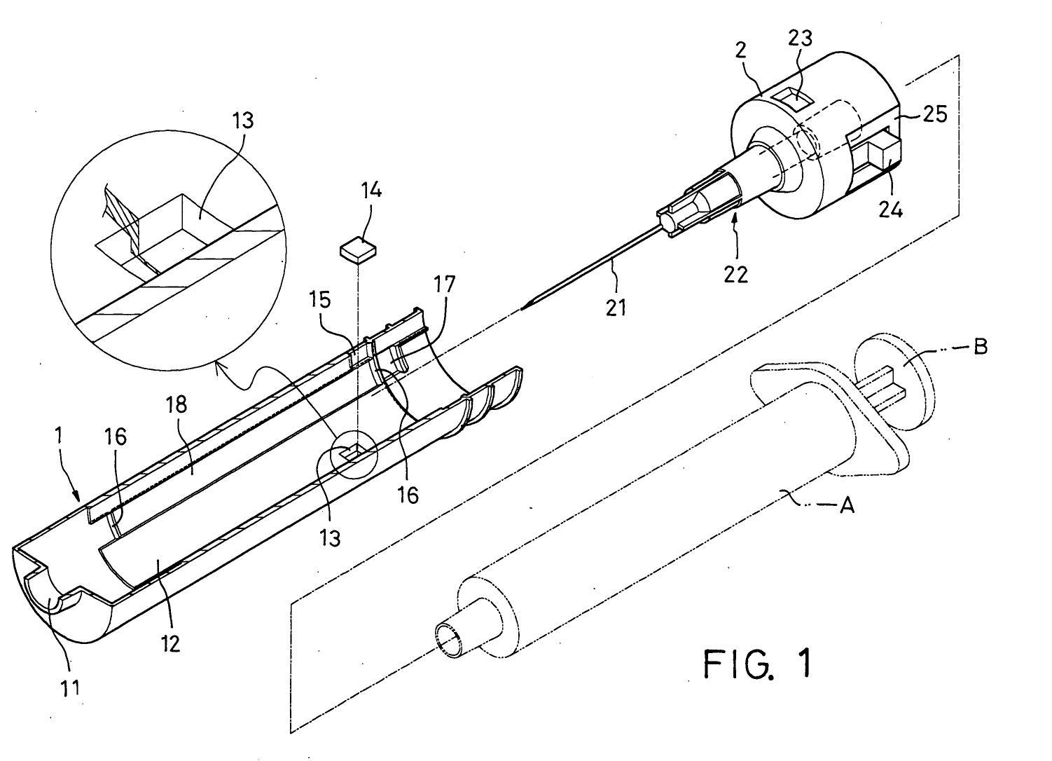

Referring to FIG. 1, the present invention is composed of a sleeve 1, a barrel A containing medicine, which is connected to a needle base 2. The functions of each component are described below:

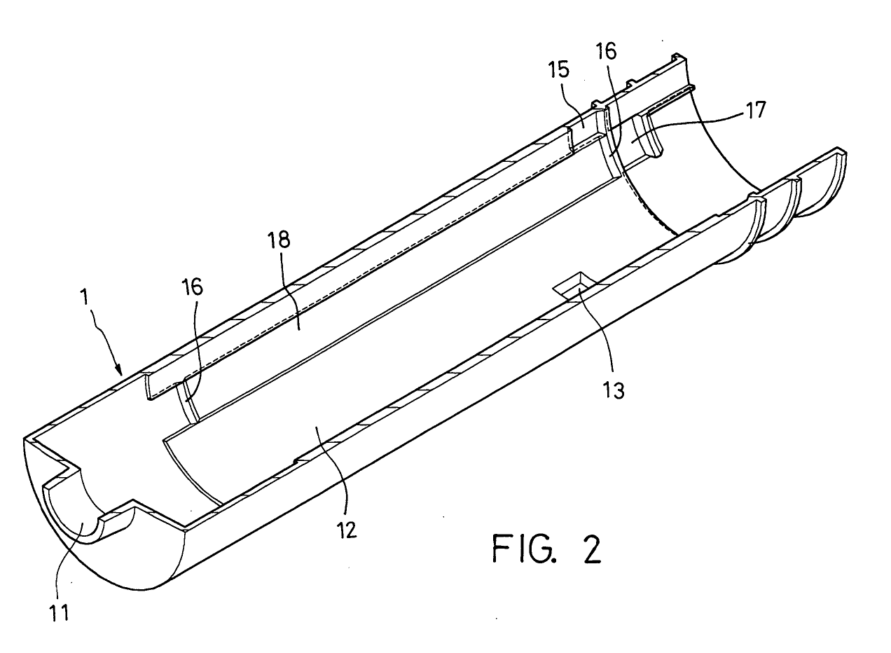

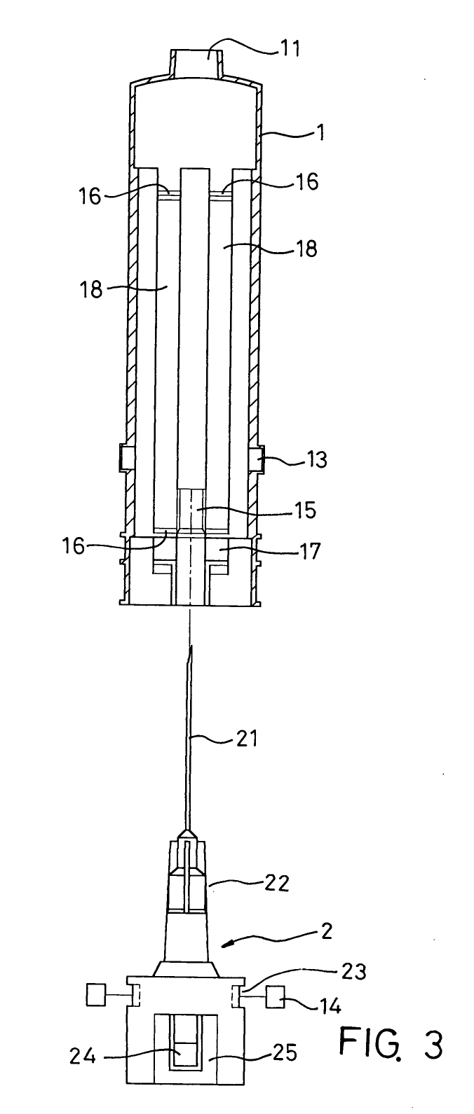

The sleeve 1, as shown from FIGS. 1 to 3, covers the needle base 2, basically, the sleeve 1 is in barrel shape, a sleeve front 11 for the needle 21 to stretch out is on the front of the sleeve 1, a chamber 12 is formed inside the sleeve 1. A pair of blocking slot 13 face to face are on the inner center of the sleeve 1, a blocking piece 14 corresponds to and is placed onto the blocking slot 13; the inner wall of the blocking slot 13 is better in slanting shape, however other different shapes is not limited; a pair of none-penetrated fastening slot 15 face to face are on the back of the inner wall of the sleeve 1, a pair of sleeve ring 16 are on the front inner wall and back inner wall of the sleeve 1, a pair of none-penetrated stopping slot 17 face to face are on the back inner wall of the sleeve 1, a sleeve path 18 spans the two sleeve ring 16.

The needle base 2 fastens the needle 21 with a needle stand 22 and connects to the barrel A, a none-penetrated base slot 23 corresponding to the blocking slot 13 is on the needle base 2, a fastening tenon 24 corresponding to the fastening slot 15 is on the flat fastening plane 25.

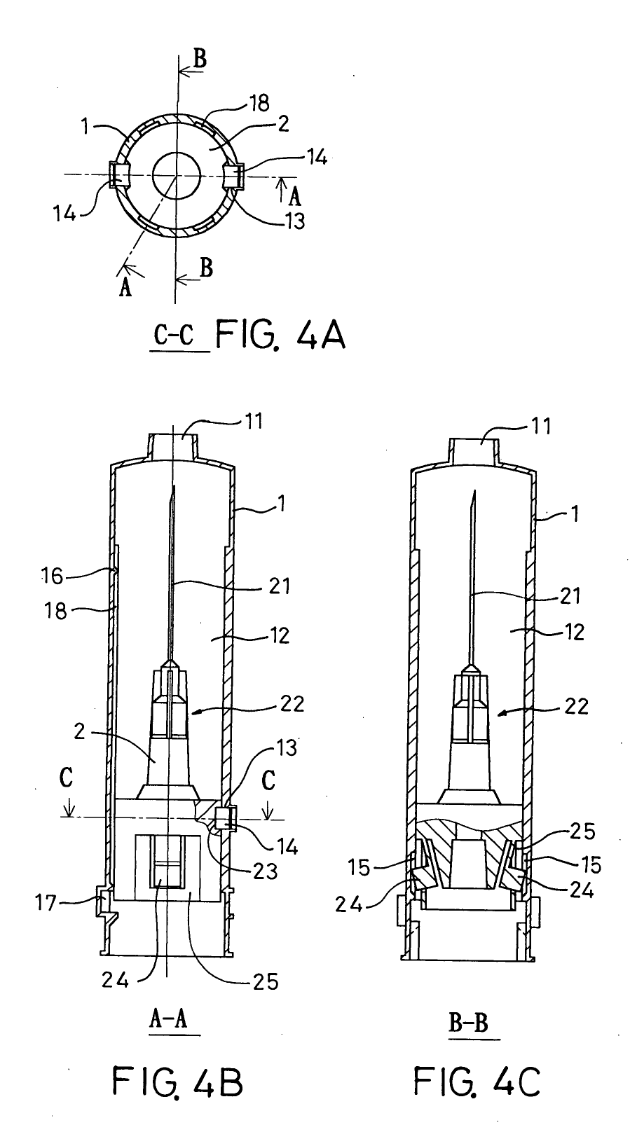

Referring to FIG. 4, while assembly, the blocking piece 14 is inserted into the blocking slot 13, at the same time the fastening tenon 24 also falls into the fastening slot 15, the blocking piece 14 is also in the base slot 23, the blocking piece 23 is inside the blocking slot 13 and the base slot 23 at the same time.

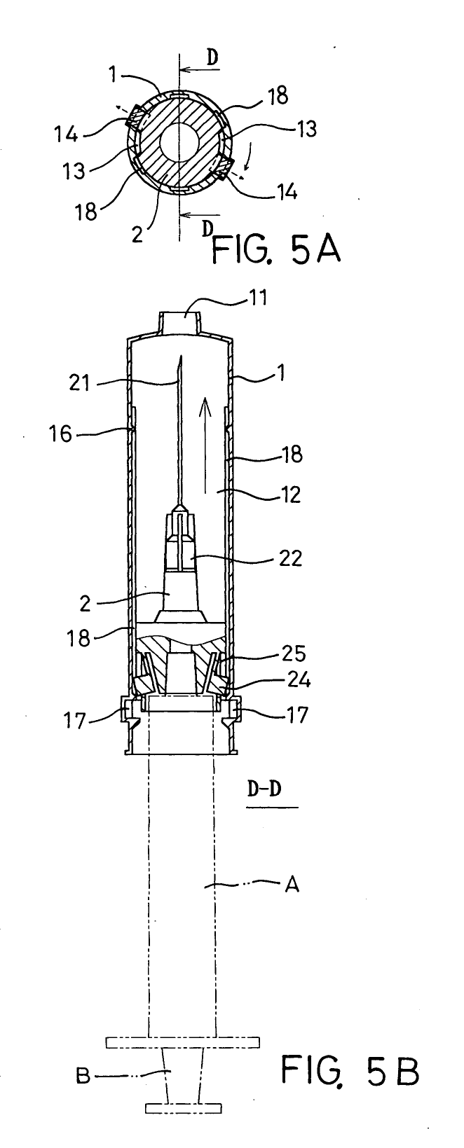

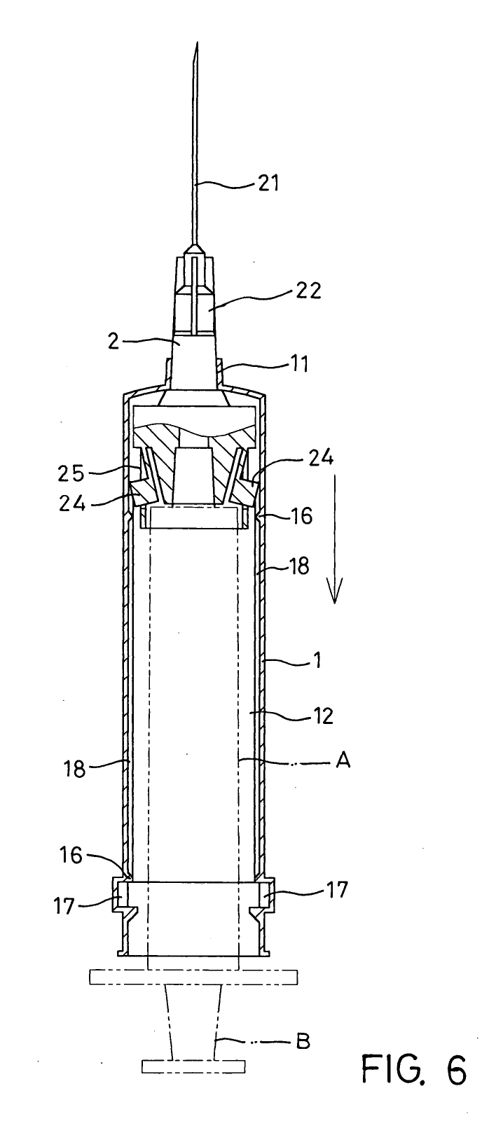

Referring to FIG. 5 shows the application, users connect the barrel A and the needle stand 22 of the needle base 2, turn certain angle to have fastening tenon 24 fall into the sleeve path 18, push needle 21 out from the sleeve front 11, pull the plunger B inside the barrel A outward to suck medicine for injection, as shown in FIG. 6. After injection, the barrel A and the plunger B are pulled backward, the fastening 24 also goes backward along the sleeve path 18, falls into stopping slot 17 and stops. The needle base 2 cannot come from the sleeve 1, the barrel A and the plunger B can get out from the needle stand 22 of the needle base 2 for recycling, the sleeve 1 and needle base 2 is in shelter state and not able to use again.

In view of the foregoing, the protective injection needle according to the present invention has the following advantages:

(1) After assembly, the sleeve and the needle base cannot be separated and remain safe during storage and transportation.

(2) The medicine drawing method is to push first then pull backward, users only have to turn a small angle and have the fastening tenon fall into sleeve path, the operation is easy and no need for training.

(3) The needle base fits all barrel sizes on the current market, also available for needles with different materials; the needle base also collocates with self-destructed needles, the cost is low, easy to operate and assemble, very safe to apply.

(4) After injection, the fixing mechanism is strong and accurate, unless with external destruction force, the needle remains inside the sleeve without exposure externally.

While a preferred embodiment of the invention has been shown and described in detail, it will be readily understood and appreciated that numerous omissions, changes and additions may be made without departing from the spirit and scope of the invention.

1. A protective injection needle comprising:
a sleeve with a sleeve front on the front end, a chamber is formed inside said sleeve, a pair of blocking slots with a corresponding blocking piece face to face are on the inner center of said sleeve, a pair of none-penetrated fastening slots face to face are on the back of the inner wall of said sleeve, a pair of sleeve rings are on the front inner wall and back inner wall of said sleeve, a pair of none-penetrated stopping slots face to face are on the back inner wall of said sleeve, a sleeve path spans two said sleeve rings;
a needle base fastens a needle with a needle stand and connects to the barrel, a none-penetrated base slot corresponding to said blocking slot is on said needle base, a fastening tenon corresponding to said fastening slot is on a flat fastening plane, while assembly, said blocking piece is inserted into said blocking slot, said fastening tenon also falls into said fastening slot, said blocking piece is in said base slot, users connect the barrel and the needle stand for medicine, turn certain angle to have said fastening tenon fall into said sleeve path, push needle out from said sleeve front, pull the plunger outward to suck medicine, after injection, the barrel and the plunger are pulled backward, said fastening also goes backward along said sleeve path, falls into said stopping slot and stops.

2. The protective injection needle recited in claim 1, wherein said blocking slot, said fastening slot and said stopping slot are installed on the same line.

3. The protective injection needle recited in claim 1, wherein said stopping slot and said sleeve path are installed on the same line.
